(19) United States
(12) Patent Application Publication
(10) Pub. No.: US 2022/0208369 A1
(43) Pub. Date: Jun. 30, 2022

(54) APPARATUS, METHOD, AND COMPUTER-READABLE STORAGE MEDIUM FOR PROVIDING HEALTH CARE SERVICE

(71) Applicant: ICON AI Inc., Seoul (KR)

(72) Inventor: Min Young Shin, Seoul (KR)

(73) Assignee: ICON AI Inc., Seoul (KR)

(21) Appl. No.: 17/564,815

(22) Filed: Dec. 29, 2021

(30) Foreign Application Priority Data
Dec. 29, 2020 (KR) 10-2020-0185548

(51) Int. Cl. G16H 40/63

(52) U.S. Cl. CPC G16H 40/63

(57) ABSTRACT

A healthcare service provision apparatus includes: a request input part which receives, from a user, a request for a biometric information measurement apparatus that measures biometric information of the user; an artificial intelligence platform service module part which recognizes an operation corresponding to the received request among operations of the biometric information measurement apparatus, and generates a control command corresponding to the recognized operation; and a transmitting part which transmits the generated control command to the biometric information measurement apparatus.

### CROSS-REFERENCE TO RELATED APPLICATION

This application is based on and claims priority to Korean Patent Application No. 10-2020-0185548, filed on Dec. 29, 2020, the application of which is incorporated herein in its entirety by reference.

### TECHNICAL FIELD

The present disclosure relates to an apparatus, method, and computer-readable storing medium for providing healthcare service.

### BACKGROUND

With the growing concerns about health, electronic devices including a biometric sensor are being developed. Such electronic devices may acquire biometric information of a human person and provide information related to the person's health using the acquired biometric information.

Normal people can easily operate these electronic devices based on an understanding of the operating principles. However, it is not easy for elderly and vulnerable people to understand the operating principles, although they have greater need to get health checkups than anyone else.

### SUMMARY

In view of this, an aspect of the present disclosure provides a technology that enables elderly and vulnerable people to easily use an electronic device that measures and provides information related to a person's health.

However, the aspects to be achieved by the present disclosure are not limited to this.

In accordance with an aspect of the present disclosure, there is provided a healthcare service provision apparatus including: a request input part which receives, from a user, a request for a biometric information measurement apparatus that measures biometric information of the user; an artificial intelligence platform service module part which recognizes an operation corresponding to the received request among operations of the biometric information measurement apparatus, and generates a control command corresponding to the recognized operation; and a transmitting part which transmits the generated control command to the biometric information measurement apparatus.

In accordance with another aspect of the present disclosure, there is provided a healthcare service provision method performed by a healthcare service provision apparatus, the method including: receiving, from a user, a request for a biometric information measurement apparatus that measures biometric information of the user; recognizing an operation corresponding to the received request among operations of the biometric information measurement apparatus; generating a control command corresponding to the recognized operation; and transmitting the generated control command to the biometric information measurement apparatus.

A healthcare service provision apparatus according to an embodiment of the present disclosure may operate such that the user themselves, their guardian, or a doctor who is in charge of their health checks the health condition of the user.

### BRIEF DESCRIPTION OF THE DRAWINGS

FIG. 1 is a block diagram of a system for providing artificial intelligence platform service and healthcare service according to an embodiment of the present disclosure.

FIGS. 2A to 3 are views showing an outward appearance of an electronic device including a healthcare service provision apparatus and a biometric information measurement apparatus according to an embodiment of FIG. 1.

FIG. 4 is a view showing an outward appearance of a sensor part of the biometric information measurement apparatus according to an embodiment of FIGS. 2A and 2B.

FIG. 5 is a view of a configuration of the healthcare service provision apparatus depicted in FIG. 1.

FIG. 6 is a view of a configuration of the biometric information measurement apparatus depicted in FIG. 1.

FIG. 7 is a flowchart of an operation for providing healthcare service according to an embodiment of the present disclosure.

### DETAILED DESCRIPTION

Advantages and features of the present disclosure, and a method of achieving them will become apparent with reference to an embodiment described later together with the accompanying drawings. However, the present disclosure is not limited to an embodiment disclosed below, but may be implemented in a variety of different forms. That is, the embodiment is provided to ensure that descriptions of the present disclosure are complete and to fully inform a scope of the invention to a person with ordinary knowledge in a technical field to which the present disclosure belongs, and the invention is only defined by the scope of claims.

In describing the embodiments of the present disclosure, if it is determined that detailed description of related known components or functions unnecessarily obscures the gist of the present disclosure, the detailed description thereof will be omitted. Further, the terminologies to be described below are defined in consideration of functions of the embodiments of the present disclosure and may vary depending on a user's or an operator's intention or practice. Accordingly, the definition thereof may be made on a basis of the content throughout the specification.

Hereinafter, an embodiment of the present disclosure will be described in detail with reference to the accompanying drawings.

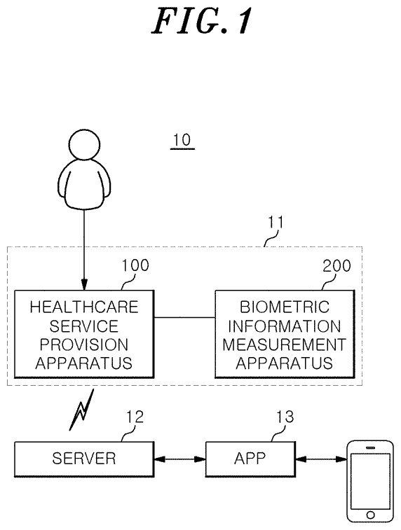

FIG. 1 is a block diagram of a system for providing healthcare service according to an embodiment of the present disclosure.

Referring to FIG. 1, the system 10 for providing health care service according to the embodiment of the present disclosure may include a healthcare service module 11 and a server 12. The health care service module 11 may be named differently as a health care service assembly, a health care device, or the like.

The healthcare service module 11 according to the embodiment of the present disclosure may include a healthcare service provision apparatus 100 and a biometric information measurement apparatus 200.

A user may be provided with their health information or biometric information through the healthcare service provision apparatus 100. To this end, upon receiving a request from the user, the healthcare service provision apparatus 100 generates a control command corresponding to this request and sends it to the biometric information measurement apparatus 200. Then, the biometric information measurement apparatus 200 performs an operation corresponding to the control command and then sends a result of the operation back to the healthcare service provision apparatus 100. Upon receiving the result, the healthcare service provision apparatus 100 may inform the user of the result or send it to a server 12 and a mobile application 13 which are illustrated in FIG. 1. Here, the mobile application 13 may be installed on a terminal and process data via network communication with the server, and may provide a display for information input and output.

Hereinafter, a module formed by combining the healthcare service provision apparatus 100 and the biometric information measurement apparatus 200 will be described.

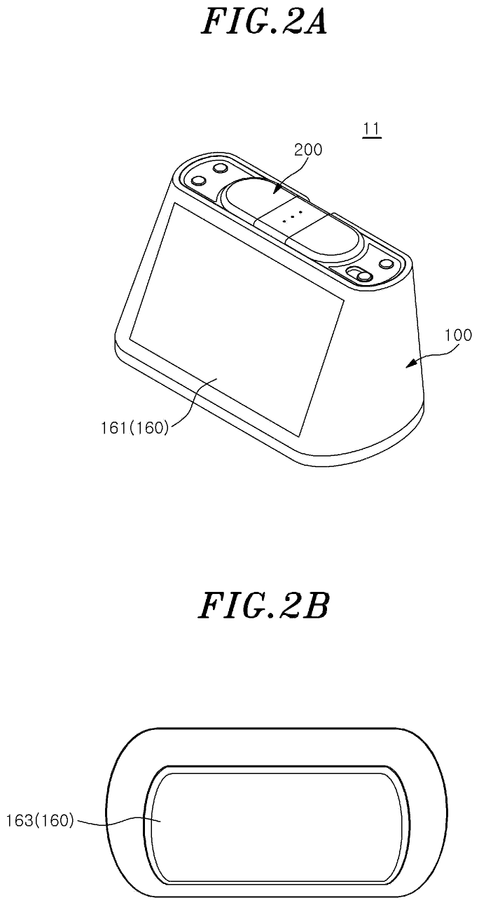

FIGS. 2A and 2B are a perspective view and bottom view of the healthcare service module 11 formed by combining the healthcare service provision apparatus 100 and the biometric information measurement apparatus 200. FIG. 3 illustrates an example in which the biometric information measurement apparatus 200 is detached from the healthcare service provision apparatus 100. However, it should be noted that FIGS. 2 and 3 are only for illustrative purposes and the spirit of the present disclosure should not be construed as being limited to what is shown in FIGS. 2A to 3.

Referring to FIGS. 2A and 2B, the biometric information measurement apparatus 200 is attached or affixed to the healthcare service provision apparatus 100. Although not depicted in FIGS. 2A and 2B, the biometric information measurement apparatus 200 may be detached from the healthcare service provision apparatus 100 as shown in FIG. 3. The user may measure their health condition or the like by means of the biometric information measurement apparatus 200 alone if detached.

Referring to FIGS. 2A and 2B, the healthcare service provision apparatus 100 and the biometric information measurement apparatus 200 may be an integrated type in which at least part of a housing of the healthcare service provision apparatus 100 and at least part of a housing of the biometric information measurement apparatus 200 are combined together, or may be an attachable and detachable type in which at least part of the housing of the healthcare service provision apparatus 100 and at least part of the housing of the biometric information measurement apparatus 200 can be attached together or detached from each other. Referring to FIG. 3, the healthcare service provision apparatus 100 may include an attachable and detachable portion 150 on top of the housing thereof, and the biometric information measurement apparatus 200 may be mounted on the attachable and detachable portion 150. The biometric information measurement apparatus 200 may include a separate power source, but may also include a rechargeable battery that is charged when seated in the healthcare service provision apparatus 100. Further, when the healthcare service provision apparatus 100 and the biometric information measurement apparatus 200 are viewed from above, the biometric information measurement apparatus 200 may have a smaller size than the top surface of the healthcare service provision apparatus 100. Further, the biometric information measurement apparatus 200 may have a size that can be carried by a user.

The healthcare service provision apparatus 100 according to the embodiment of the present disclosure includes an output part 160 that outputs a predetermined signal, and the output part 160 includes a display 161 and a speaker 163.

The output part 160 is a component that outputs a predetermined signal in response to various signals. The user may check biometric information or health information measured about himself through the output part 160.

The display 161 may be disposed on a front of the housing of the healthcare service provision apparatus 100 and provide visual information to the user, and includes one or more light-emitting diodes (LED). Visual information may be provided from an artificial intelligence platform service and shown through the display 161.

The speaker 163 may be disposed on a lower end of the housing of the healthcare service provision apparatus 100 and receive audio from the artificial intelligent platform and output it to the user.

Moreover, a microphone (not shown) may be further included to receive an audio signal from the user.

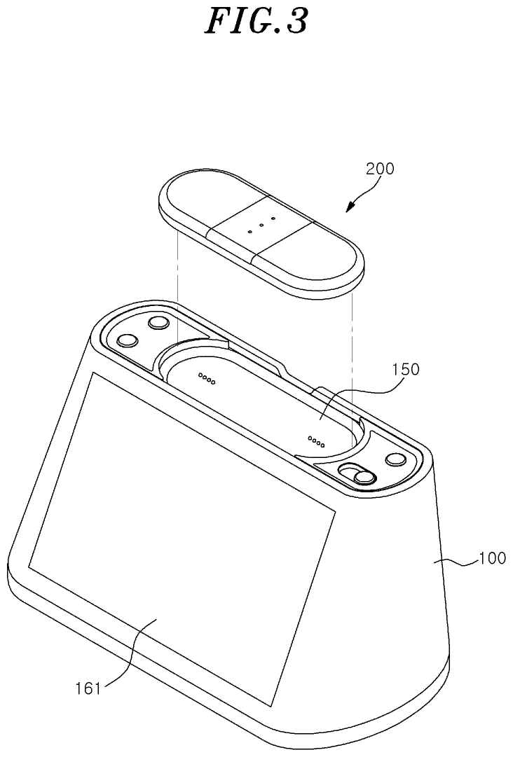

FIG. 4 is an exemplary perspective view of the biometric information measurement apparatus 200 depicted in FIGS. 2A and 2B. However, it should be noted that FIG. 4 is only for illustrative purposes, and the spirit of the present disclosure is not construed as being limited to that shown in FIG. 4.

Referring to FIG. 4, the biometric information measurement apparatus 200 includes two electrodes. One of the two electrodes is a first electrode 211, and the other electrode is a second electrode 212. Additionally, the biometric information measurement apparatus 200 may include other electrodes, apart from the first electrode 211 and the second electrode 212. For example, the biometric information measurement apparatus 200 may include a photoplethysmogram (PPG) sensor as a sensor part 210.

Among these, the PPG sensor is used to detect a PPG signal from the user, and may include an emitter and a receiver. The emitter may emit light in order to obtain data representing the user's cardiovascular condition. The light may include at least one of infrared light and visible light, and may be reflected by at least part (e.g., finger) of the user's body. The receiver may include at least one photodiode, and the at least one photodiode may receive the reflected light and convert it into an electric signal.

Further, the first electrode 211 and the second electrode 212 may be electrocardiogram (ECG) sensors, and may be used to detect an ECG signal from the user based on a touch of at least part (e.g., finger) of the user's body.

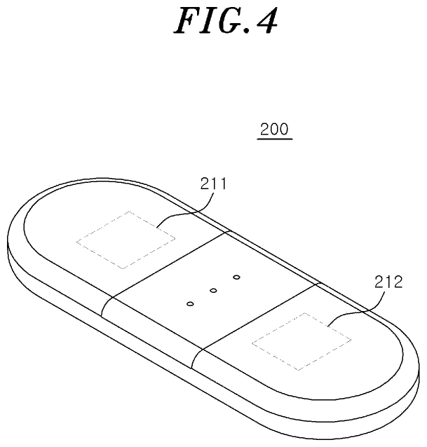

FIG. 5 is a view depicting a configuration of the healthcare service provision apparatus 100 according to the embodiment of the present disclosure. However, it should be noted that FIG. 5 is only for illustrative purposes and the spirit of the present disclosure should not be construed as being limited to that shown in FIG. 5.

First of all, the healthcare service provision apparatus 100 may be included in a product or the like that provides an artificial intelligence platform service, such as an artificial intelligent speaker as in Amazon's Alexa, or be implemented in the form of such a product, but is not limited thereto.

Accordingly, the artificial intelligence platform service applicable to an embodiment of the present disclosure may provide functions including music playback, notification settings, providing weather information, providing traffic information, and so forth.

Moreover, services may be provided based on various languages such as Korean, English, German, Japanese, French, Italian, and Hindi.

In addition, an artificial intelligence platform may operate based on a cloud to learn speech patterns and words the user use and the user's personal preferences and provide updated services.

Referring to FIG. 5, the healthcare service provision apparatus 100 may include a request input part 110, a transmitting part 120, a storage part 130, an artificial intelligence platform service module part 140, an attachable and detachable portion 150, and an output part 160, and may further include other components apart from the above components or may exclude some of the above components.

The request input part 110 is a component that is implemented to receive input from the user. The request input part 110 may be implemented by a component such as a microphone.

The transmitting part 120 is a module that performs communication, and may be implemented by a wired or wireless communication module. Through the transmitting part 120, the healthcare service provision apparatus 100 may transmit a predetermined signal to the biometric information measurement apparatus 200 or receive a predetermined signal from the biometric information measurement apparatus 200.

The storage part 130 is a means for storing data, and may be implemented by various means such as memory. The storage part 130 may store criteria for assessing whether biometric information is normal. The criteria may be numerical reference ranges.

For example, criteria for each piece of the biometric information may be stored in the form of a numerical reference range. The reference range may be a range of numerical values indicating that the user's biometric information is normal, that is, the user has no health issue.

The artificial intelligence platform service module part 140 is a module implemented to provide artificial intelligence service. The artificial intelligence platform service module part 140 may perform various functions. For example, the artificial intelligence platform service module part 140 may recognize a voice signal of the user's speech and communicate with the user in real time.

Moreover, the artificial intelligence platform service module part 140 may provide various additional services. For example, an external device electrically connected to the healthcare service provision apparatus 100 may perform a specific operation such as measuring biometric information. The specific operation may include providing the user's biometric information measured daily, weekly, and monthly, providing analyses and evaluations of the user's biometric information, providing alarm and notification information, providing particular video services, providing certain information, searching the internet, and shopping online.

The artificial intelligence platform service module part 140 may be implemented by at least one other component (e.g., hardware component or software component).

The attachable and detachable portion 150 is a component used to attach the healthcare service provision apparatus 100 and the biometric information measurement apparatus 200 together and detach them from each other. The attachable and detachable portion 150 may be implemented by a ring, latch, or magnet that is devised for attaching or detaching.

The output part 160 is a component that outputs a predetermined signal in response to various signals or the like. For example, the output part 160 may be implemented by a speaker, one or more light-emitting diodes (LEDs), etc. Through the output part 160, the user may check biometric information or health information which is measured of him.

Here, the user's biometric information or health information that can be outputted through the output part 160 may include, for example, the user's photoplethysmography, electrocardiogram, heart rate, heart rate variability, blood pressure, and/or oxygen saturation (e.g., peripheral oxygen saturation).

Hereinafter, functions that can be performed by the artificial intelligence platform service module part 140 will be described. Upon receiving a signal including the biometric information from the biometric information measurement apparatus 200 through the transmitting part 120, the artificial intelligence platform service module part 140 may check whether the biometric information deviates from a predetermined criterion which indicates the biometric information is within a normal range, by comparing the biometric information with criterion stored in the storage part 130. If the result of the comparison shows that the biometric information deviates from the predetermined criterion, the artificial intelligence platform service module part 140 may send a notification to the server 12 and mobile application 13 communicatively connected to the healthcare service provision apparatus 100.

Moreover, upon receiving a signal including biometric information, the artificial intelligence platform service module part 140 may compare the received biometric information with biometric information stored in the storage part 130. If the result of the comparison shows that a difference between the stored biometric information and the received biometric information is out of a reference range stored in the storage part 130, the artificial intelligence platform service module part 140 may decide (or determine) that the received biometric information deviates from the predetermined criterion.

In addition, if it is decided that the received biometric information deviates from the predetermined criterion, the artificial intelligence platform service module part 140 may control a first light-emitting diode of the output part 160 to emit light. If it is decided that the received biometric information does not deviate from the predetermined criterion, the artificial intelligence platform service module part 140 may control a second light-emitting diode of the output part 160 to emit light.

Furthermore, if the healthcare service provision apparatus 100 receives biometric information from the biometric information measurement apparatus 200 through the transmitting part 120, the artificial intelligence platform service module part 140 may control a third light-emitting diode of the output part 160 to emit light. As such, the user is able to get a notification of receipt of the biometric information through the artificial intelligence platform service module part 140 and the mobile application 13.

Next, the biometric information measurement apparatus 200 will be described.

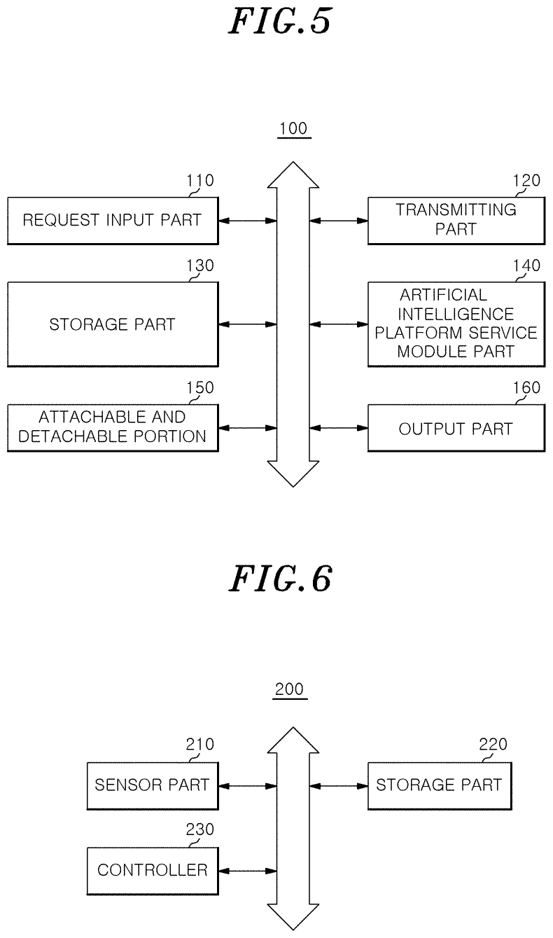

FIG. 6 is a view of a configuration of the biometric information measurement apparatus 200 according to the embodiment of the present disclosure. Referring to FIG. 6, the biometric information measurement apparatus 200 includes a sensor part 210, a storage part 220, and a controller 230. However, it should be noted that FIG. 6 is only for illustrative purposes and the spirit of the present disclosure should not be construed as being limited to that is shown in FIG. 6.

The sensor part 210 may detect (or sense) a biometric signal of the user. The biometric signal may include the user's photoplethysmography (PPG) signal (hereinafter, referred to as PPG signal) and/or electrocardiogram (ECG) signal (hereinafter, referred to as ECG signal).

The sensor part 210 may include a photoplethysmogram (PPG) sensor, a first electrode, and a second electrode. The sensor part 210 may include an analog-to-digital (ADC) converter which converts an analog signal into a digital signal.

The PPG sensor is used to detect a PPG signal from the user, and may include an emitter and a receiver. The emitter may emit light in order to obtain data representing the user's cardiovascular condition. The light may include at least one of infrared light and visible light, and may be reflected by at least part (e.g., finger) of the user's body. The receiver may include at least one photodiode, and the at least one photodiode may receive the reflected light and convert it into an electric signal.

The first electrode and the second electrode may be electrocardiogram (ECG) sensors, and may be used to detect an ECG signal from the user based on a touch of at least part (e.g., finger) of the user's body.

The storage part 220 may store various data and be implemented as memory or the like.

The controller 230 may be implemented by a microprocessor that performs various functions which is to be described below. More specifically, the controller 230 will now be described. The controller 230 receives a control command from the healthcare service provision apparatus 100 through a communication part which is not shown in the drawings, and may operate the sensor part 210 in response to the received control command or recognize a biometric signal identified by the sensor part 210.

Upon recognizing a biometric signal, the controller 230 may determine the user's biometric information based on the biometric signal, and may control the biometric information to be transmitted to the healthcare service provision apparatus 100 through the communication part not shown in the drawings.

For example, if the user's first finger makes contact with the PPG sensor and the first electrode, and the user's second finger makes contact with the second electrode, at least one biometric signal may be detected from the user through at least one of the first electrode and the second electrode under control of the controller 230. The controller 230 may determine the user's biometric information based on the detected biometric signal, and may control the biometric information to be transmitted to the artificial intelligence platform service module part 140 of the healthcare service provision apparatus 100 using the communication part not shown in the drawings.

For example, the biometric information may include the user's photoplethysmography, electrocardiogram, heart rate, heart rate variability, blood pressure, and/or oxygen saturation (e.g., peripheral oxygen saturation).

For example, the storage part 220 may store blood pressure information corresponding to each of one or more reference PPG signals, and the controller 230 may check for a reference PPG signal corresponding to the detected PPG signal, among those reference PPG signals, and determine that the blood pressure information of the reference PPG signal corresponding to the detected PPG signal is the user's blood pressure information.

For example, the controller 230 may determine information (e.g., time interval) on the user's heart rate, heart rate variability, and/or peak to peak interval (PPI) from the PPG signal.

For example, the controller 230 may determine the user's oxygen saturation based on the information on the heart rate and/or the PPI.

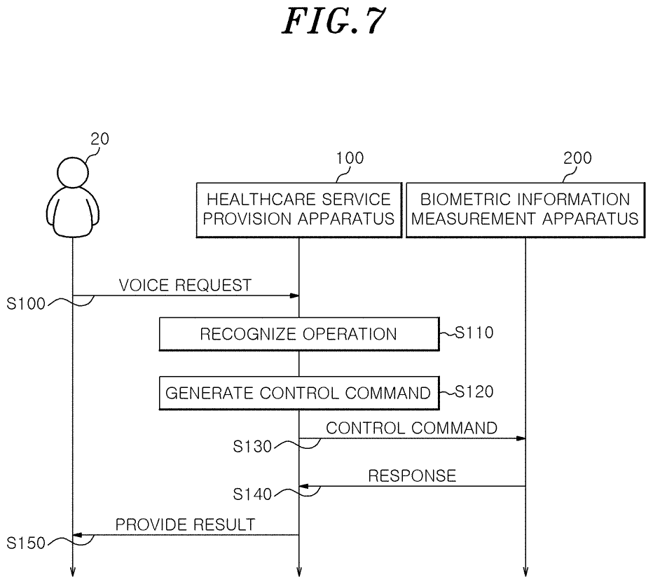

FIG. 7 is a flowchart of an operation for providing healthcare service by a healthcare service module according to the embodiment of the present disclosure.

In step S100, the healthcare service provision apparatus 100 provided in the healthcare service module 11 may receive an audio signal from the user through the request input part 110.

In step S110, the artificial intelligence platform service module part 140 provided in the healthcare service module 11 may recognize an operation of the biometric information measurement apparatus 200, corresponding to a request received in step S100, and, in step S120, may generate a control command corresponding to this operation. To this end, the storage part 130 may store one or more set reference voice commands and a control command corresponding to each of the one or more set reference voice commands. The one or more reference voice commands may include a command corresponding to a control command for activating the sensor part of the biometric information measurement apparatus 200 (or a command for turning on the power).

Next, in step S130, the healthcare service provision apparatus 100 may transmit a control command corresponding to the audio signal to the biometric information measurement apparatus 200. For example, the user's first finger may make contact with part of the PPG sensor and first electrode of the sensor part, and the user's second finger may make contact with part of the second electrode of the sensor part. Accordingly, the sensor part may detect at least one biometric information from the user. For example, the biometric signal may include a PPG signal and/or an ECG signal. Afterwards, the biometric information measurement apparatus 200 may determine the user's biometric information based on the at least one biometric signal. According to an embodiment, the biometric information measurement apparatus 200 may determine the user's photoplethysmography, electrocardiogram, heart rate, heart rate variability, blood pressure, and/or oxygen saturation (e.g., peripheral oxygen saturation), based on the PPG signal and/or the ECG signal.

Next, the biometric information measurement apparatus 200 provided in the healthcare service module 11 responds to the healthcare service provision apparatus 100 by sending a result of the operation performed by itself (step S140).

Then, the healthcare service provision apparatus 100 may control such that the biometric information is outputted using at least one of a display and a speaker or such that the biometric information is outputted to an external server, a mobile application, etc. (step S150).

In this instance, the storage part 130 of the healthcare service provision apparatus 100 may store biometric information, for example, biometric information predetermined by date (or time) of measurement, according to the user's previous operation of measuring a biometric signal.

For example, the storage part 130 of the healthcare service provision apparatus 100 may store the user's biometric information predetermined daily, weekly, and monthly. Accordingly, the healthcare service provision apparatus 100 may store the biometric information received according to the above operations and statistics of the stored biometric information.

According to an embodiment, the healthcare service provision apparatus 100 may check whether the received biometric deviates from a predetermined criterion, for example, a normal range (a data range of a specific scope), by comparing the biometric information received according to the above operations and the predetermined and stored biometric information. The normal range may be determined based on the stored biometric information. Once the received biometric information is found to deviate from the normal range, the healthcare service provision apparatus 100 may send a notification to preset devices to indicate that the received biometric information deviates from the normal range. For example, the preset devices may include the server 12 of FIG. 1. Through this, if the user (or owner) of the healthcare service provision apparatus 100 is a relative of the user or a senior, the healthcare service provision apparatus 100 allows their child to get the notification automatically through a means such as a mobile application or the like, or, if the user (or owner) of the healthcare service provision apparatus 100 is a handicapped person, it allows a hospital (or nurse) and/or an emergency room to get the notification automatically through means such as a mobile application or the like.

According to the embodiment, the healthcare service provision apparatus 100 may periodically send the user's biometric information to a designated server (e.g., a server of a designated hospital) based on the user's settings, i.e., with the consent of the user. Accordingly, the user's doctor in the designated hospital may frequently check the user's health condition and the user's doctor may send information such as health care tips for the user to the healthcare service provision apparatus 100 by using an electronic device. If the user's doctor determines that an abnormal symptom has occurred to the user, the user's doctor may send information to the healthcare service provision apparatus 100 to inform that the user should visit a hospital, by using an electronic device.

According to the embodiment, the healthcare service provision apparatus 100 may provide women who are on a diet with information, such as an analysis of body fat as in InBody, or provide them with big data and health management functions.

According to the embodiment, when the healthcare service provision apparatus 100 stores the biometric information in the storage part 130, the biometric information may be stored in such a way as to correspond to user information. Thus, the storage part 130 may store biometric information corresponding to each user. For example, the healthcare service provision apparatus 100 may build big data regarding the process of change of biometric information, to fit with the lifecycle of each individual user, and manage personalized healthcare information (or health data) based on the big data.

In addition to the above-described embodiment, the one or more preset reference voice commands may further include a command corresponding to a control command for outputting a pre-stored user guide on the display of the healthcare service provision apparatus 100 and/or outputting the pre-stored user guide through a speaker. For example, if the user utters a command corresponding to a control command for outputting the pre-stored user guide, the healthcare service provision apparatus 100 may receive an voice signal corresponding to the command using a microphone and therefore output the pre-stored user guide. For example, the pre-stored user guide is for providing healthcare service, and may include visual information and/or voice information that urges the user to touch their finger on the sensor part for a specified amount of time.

According to the foregoing embodiments, the healthcare service provision apparatus 100 according to the embodiment of the present disclosure may determine whether basic biometric information of the user who owns the healthcare service provision apparatus 100 and the biometric information measurement apparatus 200, for example, the user's photoplethysmography, electrocardiogram, heart rate, heart rate variability, blood pressure, and/or oxygen saturation (e.g., peripheral oxygen saturation), deviates from a predetermined reference range (standard range), rather than providing telemedicine in a precise manner by which a prescription is practically given for a certain disease. If it is determined that the biometric information deviates from the predetermined reference range, the healthcare service provision apparatus 100 may provide a designated notification through the display and/or the speaker and/or the mobile application. The designated notification may include a guidance that urges the user to visit the doctor immediately or within a few days and/or a reference and basic guide that provides a simple and basic prescription and health management information. Or else, the healthcare service provision apparatus 100 may send the biometric information to a particular server, and the particular server may determine that the biometric information deviates from the predetermined reference range and provide telemedicine in which the notification is sent to the healthcare service provision apparatus 100 so that the user of the healthcare service provision apparatus 100 reads the notification. For example, in countries that suffer from a lack of medical facilities and offer low levels of medical coverage, and where medical costs are high, an electronic device including the healthcare service provision apparatus 100 and the biometric information measurement apparatus 200 according to the embodiment of the present disclosure may come in useful. For example, if an electronic device including the healthcare service provision apparatus 100 and the biometric information measurement apparatus 200 according to the embodiment of the present disclosure is provided to a user who can't afford in-person visits to a doctor because of the burden of medical expenses or due to a lack of transport or long travel time, the user may be given a simple prescription such as aspirin, or may be advised to visit a doctor as soon as possible in case of an emergency, through the electronic device. That is, an electronic device including the healthcare service provision apparatus 100 and the biometric information measurement apparatus 200 according to the embodiment of the present disclosure may be used and implemented as an aid for telemedicine.

Various embodiments of the present disclosure may be implemented as software (e.g., program) including instructions stored in machine (e.g., computer) readable storage media (e.g., memory 215 (internal memory or external memory)). The machine is a device that can invoke the stored instructions from the storage media and operate in accordance with the invoked instructions, and may include an electronic device (e.g., electronic device 200) according to the disclosed embodiments. When the command is executed by a processor (e.g., processor 201), the processor may perform functions corresponding to the instructions, either directly or by using other components under the control of the processor. The instructions may include code generated by a compiler or executable by an interpreter. The machine readable storage media may be provided in the form of non-transitory storage media. Here, the term ‘non-transitory’ means that the storage media do not include a signal and is tangible, but do not distinguish whether data is stored semi-permanently or temporarily in the storage media.

According to the embodiment of the disclosure, a method according to various embodiments disclosed in the present disclosure may be included and provided in a computer program product.

The above description is merely exemplary description of the technical scope of the present disclosure, and it will be understood by those skilled in the art that various changes and modifications can be made without departing from original characteristics of the present disclosure. Therefore, the embodiments disclosed in the present disclosure are intended to explain, not to limit, the technical scope of the present disclosure, and the technical scope of the present disclosure is not limited by the embodiments.

Therefore, the protection scope of the present disclosure should be interpreted based on the following claims and it should be appreciated that all technical scopes included within a range equivalent thereto are included in the protection scope of the present disclosure.

What is claimed is:

1. A healthcare service provision apparatus comprising:
a request input part which receives, from a user, a request for a biometric information measurement apparatus that measures biometric information of the user;
an artificial intelligence platform service module part which recognizes an operation corresponding to the received request among operations of the biometric information measurement apparatus, and generates a control command corresponding to the recognized operation; and
a transmitting part which transmits the generated control command to the biometric information measurement apparatus.

2. The healthcare service provision apparatus of claim 1, further comprising:
an attachable and detachable portion having a function to provide attachment and detachment between the biometric information measurement apparatus and the healthcare service provision apparatus.

3. The healthcare service provision apparatus of claim 1, wherein the received request includes at least one of a request that causes the biometric information measurement apparatus to measure biometric information of the user, a request to output the measured biometric information of the user to the healthcare service provision apparatus, a request to send the measured biometric information of the user to a predetermined destination, a request that causes the biometric information measurement apparatus to delete the measured biometric information of the user, and a request about how to deal with the measured biometric information of the user when the biometric information deviates from a predetermined criterion.

4. The healthcare service provision apparatus of claim 3, wherein the request that causes the biometric information measurement apparatus to measure the user's biometric information includes:
a request to measure at least one of heart rate, heart rate variability, blood pressure, peripheral oxygen saturation, photoplethysmography, and electrocardiogram.

5. The healthcare service provision apparatus of claim 3, wherein the request about how to deal with the measured biometric information of the user when the measured biometric information deviates from the predetermined criterion includes:
a request to send a notification to a server or mobile application communicatively connected to the healthcare service provision apparatus.

6. A healthcare service provision method performed by a healthcare service provision apparatus, the method comprising:
receiving, from a user, a request for a biometric information measurement apparatus that measures biometric information of the user;
recognizing an operation corresponding to the received request among operations of the biometric information measurement apparatus;
generating a control command corresponding to the recognized operation; and
transmitting the generated control command to the biometric information measurement apparatus.

7. The healthcare service provision method of claim 6, wherein the received request includes at least one of a request that causes the biometric information measurement apparatus to measure biometric information of the user, a request to output the measured biometric information of the user to the healthcare service provision apparatus, a request to send the measured biometric information of the user to a predetermined destination, a request that causes the biometric information measurement apparatus to delete the measured biometric information of the user, and a request about how to deal with the measured biometric information of the user when the biometric information deviates from a predetermined criterion.

8. The healthcare service provision method of claim 7, wherein the request that causes the biometric information measurement apparatus to measure the user's biometric information includes:
a request to measure at least one of heart rate, heart rate variability, blood pressure, peripheral oxygen saturation, photoplethysmography, and electrocardiogram.

9. The healthcare service provision apparatus of claim 7, wherein the request about how to deal with the measured biometric information of the user when the measured biometric information deviates from the predetermined criterion incudes:
a request to send a notification to a server or mobile application communicatively connected to the healthcare service provision apparatus.

10. A non-transitory computer readable storage medium storing a computer program, programmed to perform the method of claim 6 by a processor.
